(19) United States
(12) Patent Application Publication
(10) Pub. No.: US 2017/0071524 A1
(43) Pub. Date: Mar. 16, 2017

(54) METHOD OF CORRECTING DISTORTION OF PSYCHOLOGICAL TEST USING USER'S BIOMETRIC DATA

(71) Applicant: GRIT Research Institute, Seoul (KR)

(72) Inventors: Youngsan YOO, Seoul (KR); Joohan KIM, Seoul (KR)

(73) Assignee: GRIT Research Institute, Seoul (KR)

(21) Appl. No.: 15/263,863

(22) Filed: Sep. 13, 2016

(30) Foreign Application Priority Data
Sep. 14, 2015 (KR) 10-2015-0129656

(51) Int. Cl. A61B 5/16; A61B 5/0402; A61B 5/00; A61B 5/11; A61B 5/145; A61B 5/0205; G06F 19/00; A61B 5/0476

(52) U.S. Cl. CPC A61B 5/165; G06F 19/366; A61B 5/0402; A61B 5/0476; A61B 5/11; A61B 5/14546; A61B 5/02055; A61B 5/4261; A61B 5/4519; A61B 5/6801; A61B 2562/0219; A61B 5/02416

(57) ABSTRACT

Disclosed is a method of correcting distortion of a psychological test using a user's biometric data. By using the method of correcting distortion of a psychological test, it is possible to correct a score of a user who makes response distortion and provide a better selection that meets the objective of the psychological test. Also, it is also possible to train an item with a response instinctually distorted by the user through an analysis of result data of the psychological test training program, thus decreasing the response distortion of the user.

### RELATED APPLICATION

This application claims the benefit of Korean Patent Application No. 10-2015-0129656, filed on Sep. 14, 2015, in the Korean Intellectual Property Office, the disclosure of which is incorporated herein in its entirety by reference.

### BACKGROUND

1. Field

One or more exemplary embodiments relate to a method of correcting distortion of a psychological test using a user's biometric data, and more particularly, to a method of correcting distortion of a psychological test by extracting a question item with a response distorted by the user from among question items for the psychological test and adjusting a response score assigned to the extracted question item.

2. Description of the Related Art

Among psychological tests, personality tests are used by companies to select human resources appropriate for their organizations and duties. Generally, personality tests are test methods of presenting one adjective or sentence and allowing self-estimation of how well individual internal attributes (e.g., traits, motives, and interests), which lead to a consistent behavior, are represented. Most of the personality tests may be normative type tests.

In the normative type, test candidates has to rate how strongly they agree on a scale of 3-7 in a Likert scale (which is one of scales widely used to digitize an attitude or value). An individual response is summed up to a total score. However, the total score does not represent an absolute level of any characteristic because the total score is a raw score. Accordingly, the total score is interpreted as a relative score based on other people's scores and is used to perform estimation through interpersonal comparison.

However, the normative type is exposed to a problem of response distortion. The response distortion is a kind of impression management for which candidates intentionally distort their responses to make positive images. The response distortion may include a central tendency, which is the tendency to cause response distortion in order to avoid an extreme range; a lenience tendency, which is the tendency to cause response distortion in order to overestimate their competence regardless of actual performance; an acquiescence, which is the tendency to fully agree without any thought whatsoever; and a strictness tendency, which is the tendency to underestimate themselves due to their low self-esteem.

Since candidates attempt to distort responses in a socially desirable manner in order to be selected, such response distortion makes it not easy to guarantee reliability in an employment process using a personality test. Accordingly, human resources that are not appropriate to an organization and duties may be incorrectly selected.

Accordingly, due to the limitations in using the existing personality tests, there is a strong need for an alternative solution for correcting the response distortion to select human resources that are appropriate for an organization and duties.

### SUMMARY

In order to solve the above problems, according to an embodiment of the present invention, a method of correcting distortion of a psychological test using a user's biometric data and a psychological test training program is intended to be provided.

Accordingly, it is possible to correct a score of a user who makes response distortion and provide a better selection that meets the objective of the psychological test, and it is also possible to train an item with a response instinctually distorted by the user through an analysis of result data of the psychological test training program, thus decreasing the response distortion of the user.

Additional aspects will be set forth in part in the description which follows and, in part, will be apparent from the description, or may be learned by practice of the presented exemplary embodiments.

According to an embodiment of the present invention, a method of correcting distortion of a psychological test may be provided.

According to one or more exemplary embodiments, a method of correcting distortion of a psychological test using a user's biometric data includes receiving personal information for authentication from the user and performing user authentication; acquiring first biometric data from the user at predetermined intervals while the user is in a normal state; allowing the user to perform a psychological test training program and acquiring second biometric data from the user of the program at predetermined intervals; comparing the first biometric data and the second biometric data to acquire third biometric data, which is reference data for distortion detection; allowing the user to take the psychological test and acquiring fourth biometric data from the user at predetermined intervals; comparing the third biometric data and the fourth biometric data; analyzing a result of the psychological test of the user based on a result of the comparison; and correcting the result of the psychological test based on a result of the analysis.

The personal information may include user identification information composed of at least one of text, image, and sound. The normal state of the user may include a state in which the user engages in daily life. The predetermined intervals may be regular intervals of one millisecond, one second, or one minute.

The personal information, the first biometric data, the second biometric data, the third biometric data, and the fourth biometric data may be received or acquired through a user device. The user device may include at least one of a data input unit and a sensor unit. The sensor unit may include a photoplethysmography (PPG) sensor, an electrocardiography (ECG) sensor, an electroencephalogrpahy (EEG) sensor, a galvanic skin response (GSR) sensor, a skin temperature (SKT) sensor, an accelerator, a gyro sensor, or a cortisol hormone measurement sensor.

Each of the personal information, the first biometric data, the second biometric data, the third biometric data, and the fourth biometric data may include at least one of a pulse rate, a respiration rate, secreted body fluids, a pupil size, a body temperature, and muscle momentum of the user.

The psychological test training program may be a program that sequentially outputs at least one of image and sound according to a psychological state of the user. The program may be temporarily or regularly performed by the user. The psychological state of the user may be determined according to current state information entered by the user.

The comparing of the third biometric data and the fourth biometric data may include estimating a degree of consistency between the third biometric data and the fourth biometric data. The analyzing of a result of the psychological test of the user based on a result of the comparison may include extracting a psychological test item that has been answered while the fourth biometric data inconsistent with the third biometric data is measured.

The correcting of the result of the psychological test based on a result of the analysis may include performing a calculation between a score of response of the user to the extracted psychological test item and a correction factor to adjust the response score. The psychological test may include a plurality of incompatible items or at least one item that reflects an index for estimating a response pattern of a user who makes responses distorted to conform to social norms.

According to an embodiment of the present invention, a non-transitory computer-readable recording medium having a program for executing the above-mentioned method stored therein may be provided.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 is a flowchart showing a method of correcting distortion of a psychological test using a user's biometric data according to an embodiment of the present invention;

FIG. 2 is a view showing an organic connection relationship among a user, a user device, a psychological test device, and a database (DB) server in a method of correcting distortion of a psychological test using the user's biometric data according to an embodiment of the present invention;

FIG. 3 is a view showing a series of exemplary processes for selecting a final successful candidate by various kinds of institutions through a method of correcting distortion of a psychological test using a user's biometric data according to an embodiment of the present invention; and

FIG. 4 is a view showing a process of estimating distortion of a psychological test using a user's biometric data according to an embodiment of the present invention.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, embodiments of the present invention will be described in detail to be easily embodied by those skilled in the art with reference to the accompanying drawings. The present invention may, however, be embodied in many different forms and should not be construed as being limited to the embodiments set forth herein. In the accompanying drawings, portions irrelevant to a description of the exemplary embodiments will be omitted for clarity. Moreover, like reference numerals refer to like elements throughout.

The terms used herein will be briefly described, and the present invention will be described in detail.

Terms used in the present invention have been selected as general terms which are widely used at present, in consideration of the functions of the present invention, and this may be altered according to the intent of an operator skilled in the art, conventional practice, or introduction of new technology. Also, if there is a term which is arbitrarily selected by the applicant in a specific case, in this case, a meaning of the term will be described in detail in a corresponding description portion of the exemplary embodiments. Therefore, the terms used herein should be defined on the basis of the overall content of the present invention instead of a simple name of each of the terms.

Furthermore, when one part is referred to as “comprising (or including or having)” other elements, it should be understood that it can comprise (or include or have) only those elements, or other elements as well as those elements unless specifically described otherwise. Moreover, each of terms such as “unit” and “module” described in the embodiments denotes an element for performing at least one function or operation, and may be implemented in hardware, software or a combination of hardware and software. In this disclosure below, when one part (or element, device, etc.) is referred to as being “connected” to another part (or element, device, etc.), it should be understood that the former can be “directly connected” to the latter, or “electrically connected” to the latter via an intervening part (or element, device, etc.).

Hereinafter, embodiments of the present invention will be described in detail with reference to the accompanying drawings.

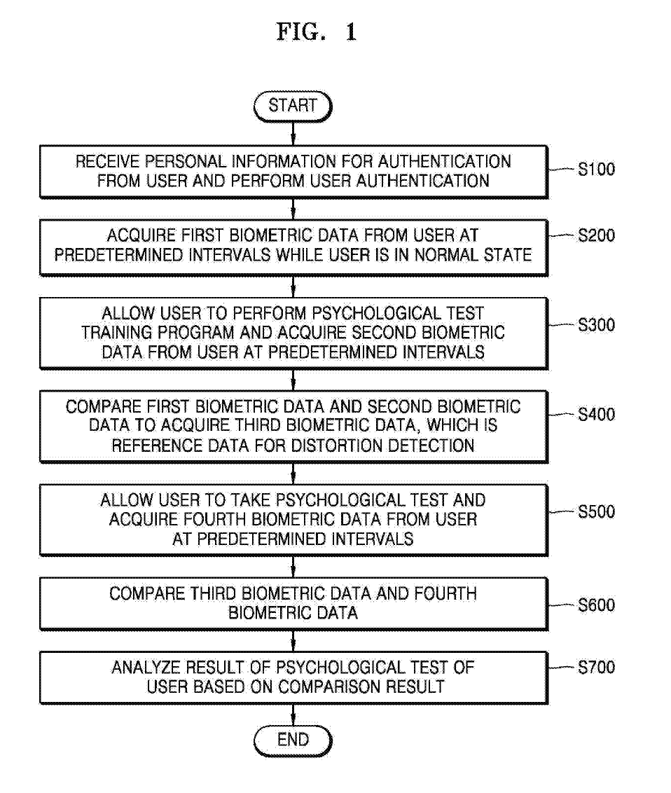

FIG. 1 is a flowchart showing a method of correcting distortion of a psychological test using a user's biometric data according to an embodiment of the present invention.

The method of correcting distortion of a psychological test using a user's biometric data according to an embodiment of the present invention may include receiving personal information for authentication from the user and perform user authentication (S100); acquiring first biometric data from the user at predetermined intervals while the user is in a normal state (S200); allowing the user to perform a psychological test training program and acquiring second biometric data from the user of the program at predetermined intervals (S300); comparing the first biometric data and the second biometric data to acquire third biometric data, which is reference data for distortion detection (S400); allowing the user to take the psychological test and acquiring fourth biometric data from the user at predetermined intervals (S500); comparing the third biometric data and the fourth biometric data (S600); and analyzing a result of the psychological test of the user based on a result of the comparison (S700). In addition, the distortion correction method may further include correcting the result of the psychological test based on a result of the analysis that is acquired in the above-described method.

The psychological test may include, for example, the GRIT test, a character trait test, an intelligence test, an aptitude test, or a simple neuropsychological test, but is not limited thereto as long as the test uses a Likert scale.

Furthermore, the character trait test may include the Myers-Briggs Type Indicator (MBTI), the Murphy-Meisgeier Type Indicator for Children (MMTIC), and the Minnesota Multiphasic Personality Inventory (MMPI). The Minnesota Multiphasic Personality Inventory (MMPI) is a self-reported test that was developed to efficiently and reliably diagnose and estimate psychopathology of patients in clinical disciplines of neuropsychiatry. An original MMPI, an MMP-2 for adults, or an MMPI-A for adolescents may be used as a diagnostic assessment tool and also may be included in order to quantitatively measure various psychological research areas such as psychological counseling, personnel selection, legal advices, or selection tests or researches related to mental health.

Also, the personal information may include user identification information composed of at least one of text, image, and sound. The normal state of the user may include a state in which the user engages in daily life. The predetermined intervals may regular intervals of one millisecond, one second, or one minute. The text may be specific characters or text prestored in the DB. For the user authentication, the user may write down specific characters or text provided by a psychological test distortion correction system. The user authentication may be performed on the basis of an analysis of a style of the text written down by the user.

Also, the image may be an image of the user's face. For example, the user may allow the user authentication to be performed by photographing his or her face with a camera and delivering an image of the photographed face to a psychological test distortion correction system. The psychological test distortion correction system may perform the user authentication by analyzing similarity between the image provided by the user and a user face image prestored in the DB.

Also, the sound may be the user's voice. For example, when the user transfers his or her voice to the psychological test distortion correction system, the system may perform the user authentication through a voice print analysis between the user's voice and a user voice prestored in the DB. In other words, the user authentication may be performed on the basis of a result of the voice print analysis. The above-described user face image and user voice may be acquired through a user device having a photographing function and a voice collecting function. For example, the user face image may be acquired through a camera of the user device, and the user voice may be acquired through a microphone of the user device.

Also, the predetermined intervals may be regular intervals of one millisecond, one second, one minute, or ten minutes.

The psychological test training program may include a GRIT training program. The GRIT training program consecutively or repetitively outputs at least one of image and sound according to a psychological state of a user. The user may train a non-cognitive ability by watching media content that is consecutively or repetitively output and performing bidirectional communication.

Also, the second biometric data may acquire an average rate of changes of values measured at predetermined intervals after a first question item of the psychological test training program.

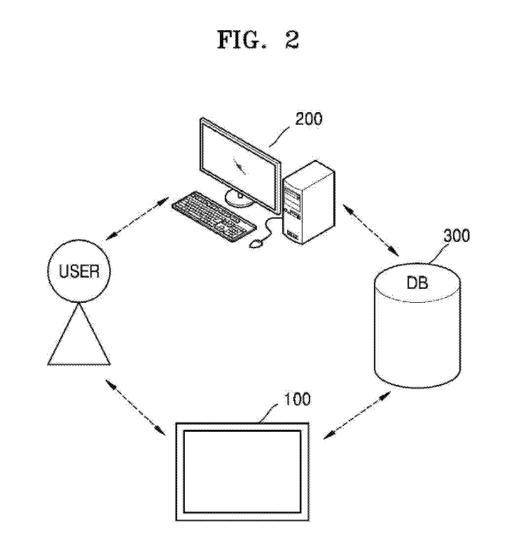

FIG. 2 is a view showing an organic connection relationship among a user, a user device 100, a psychological test device 200, and a DB server 300 in the method of correcting distortion of a psychological test using a user's biometric data according to an embodiment of the present invention.

In the method of correcting distortion of a psychological test using a user's biometric data according to an embodiment of the present invention, the personal information and the first to fourth biometric data may be received or acquired through the user device 100. The user device 100 may be a personal desktop computer (PC), a tablet PC, a laptop, or a smartphone, but is not limited thereto. In other words, the user device 100 may be a device capable of wireless communication with other devices. In addition, the user device 100 may be a wearable device including at least one sensor unit. For example, the user device 100 may be a smart watch.

The user device 100 may include at least one of a data input unit (not shown) and a sensor unit (not shown). The sensor unit (not shown) may include a photoplethysmography (PPG) sensor, an electrocardiography (ECG) sensor, an electroencephalogrpahy (EEG) sensor, a galvanic skin response (GSR) sensor, a skin temperature (SKT) sensor, an accelerator, a gyro sensor, or a cortisol hormone measurement sensor, but is not limited thereto as long as it can measure a ballistocardiogram (BCG), a bladder pressure, a blood flow, a blood pressure, a blood gas, a blood pH, a cardiac output, an electroencephalogram, an electrogastrogram, an electromyography, an electrooculogram, a gastric acidity, a gastric pressure, a gastric force, a nerve potential, or a phonocardiogram (PSG) of a user.

Also, the first biometric data measured by the sensor unit (not shown) may be utilized as the user's personal information.

Also, the personal information and the first to fourth biometric data may include at least one of a pulse rate, a respiration rate, secreted body fluids, a pupil size, a body temperature, and muscle momentum of the user, but is not limited thereto as long as a difference in a biometric signal between the user's normal state and the user's tension or excitation state may occur. The biometric signal may be usefully used as the personal information in that the biometric signal can be used only while the user is alive and also cannot be used when the biometric signal is affected by an emotional change and a physical change due to a threat.

Also, the personal information and the first to fourth biometric data may be stored in the DB server 300.

Also, the psychological test training program is a program that consecutively outputs at least one of image and sound according to a psychological state of the user. The program may be temporally or regularly performed by the user, and the user's psychological state may be determined according to information about the current state of the user that is input from the user.

Also, the psychological test training program may include a GRIT training system (GTS) and may be performed through the psychological test device 200. The psychological test device 200 may be a personal desktop computer (PC), a tablet PC, a laptop, or a smartphone, but is not limited thereto. In other words, the psychological test device 200 may be a device capable of wireless communication with other devices. In addition, the psychological test device 200 may be a wearable device including at least one sensor unit such as a smart watch.

Also, data about a training program result obtained by the user performing the psychological test training program may be stored in the DB server 300.

Also, the user may perform user authentication through personal information input through an input unit (not shown) of the user device 100 or personal information obtained through the sensor unit (not shown). Such authentication is intended to prevent cheating on the test. Accordingly, the user authentication may be repeatedly performed at least one or more times while the user takes the psychological test as well as when the user intends to initiate the psychological test. For example, while a user takes a psychological test, a request to enter personal information for user authentication may be delivered to the user through a psychological test distortion correction system. When the request to enter personal information is received, the user may enter unique personal information to allow the user authentication to be successfully performed even while the user takes the psychological test.

Also, user-specific biometric data may be used for the user authentication. For example, the personal information for the user authentication may include a pulse rate, a respiration rate, secreted body fluids, a pupil size, a body temperature, and muscle momentum, which are unique to the user. Also, a change in the pulse rate, the respiration rate, or the body temperature of the user for each season or for each time may be utilized as the personal information for the user authentication. The user-specific biometric data may be acquired through a user device such as a wearable device in real time, and the acquired biometric data may be utilized for the user authentication.

For example, when a user removes the wearable device while taking a psychological test, continuity of collection of the user's biometric data is lost. In this case, the psychological test distortion correction system may deliver a request to enter personal information for user authentication to the user. When a user re-wears the wearable device, the psychological test distortion correction system may compare biometric data acquired before the re-wearing and biometric data acquired after the re-wearing and perform user authentication in order to determine whether the user is the same as before. When there is a large difference between the biometric data before the re-wearing and the biometric data after the re-wearing, the user authentication may fail.

For a certain time while the user authentication is performed, first biometric data may be acquired through a sensor unit (not shown). A user may perform a psychological test training program through the psychological test device 200. Second biometric data may be acquired through a sensor unit (not shown) for each psychological test item of the training program for a certain time. The first biometric data and the second biometric data acquired through the sensor unit (not shown) may be stored in the DB server 300. The first biometric data and the second biometric data stored in the DB server 300 are compared with each other to acquire third biometric data, which is biometric data that is a criterion of whether there is response distortion in the psychological test training program performed by the user. The third biometric data may be stored in the DB server 300. Accordingly, the user may not take a psychological test after completing the psychological test training program. Last, while the user takes the psychological through the psychological test device 200, fourth biometric data may be acquired for each psychological test item through a sensor unit (not shown) for a certain time. The acquired fourth biometric data may be stored in the DB server 300. An item with a distorted response may be extracted by comparing the fourth biometric data with the third biometric data. For the extracted response-distorted item, a final score may be acquired by performing a calculation between a score of response of the user to the item and a correction factor to adjust the response score. The acquired final score may be stored in the DB server 300, and a ranking may be determined by comparing final scores of users who took the psychological test.

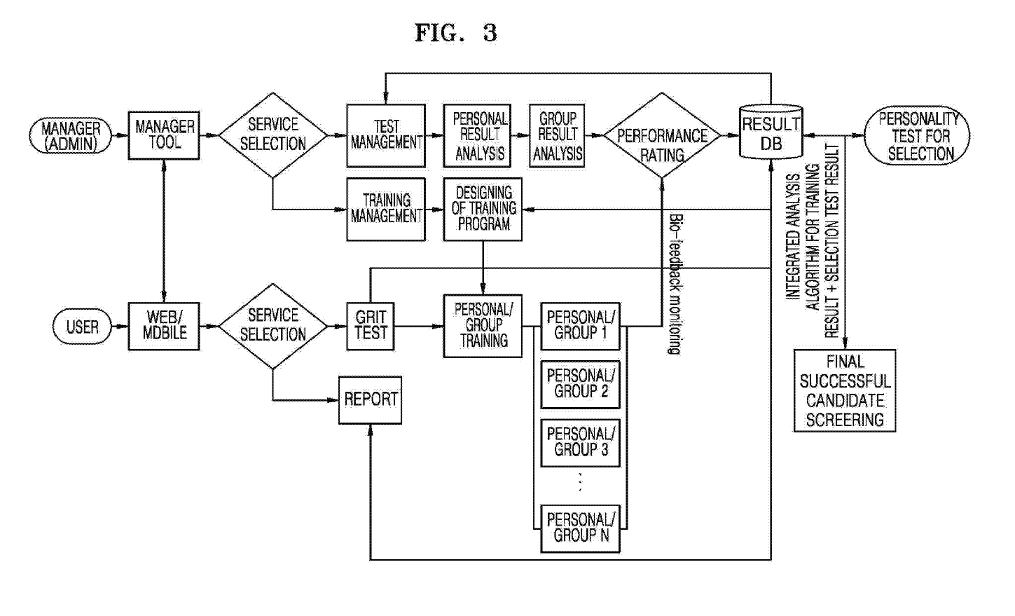

FIG. 3 is a view showing a series of exemplary processes for selecting a final successful candidate by various kinds of institutions through a method of correcting distortion of a psychological test using a user's biometric data according to an embodiment of the present invention.

In the method of correcting distortion of a psychological test using a user's biometric data according to an embodiment of the present invention, a step of comparing third biometric data and fourth biometric data may include estimating a degree of consistency between the third biometric data and the fourth biometric data, and a step of analyzing a psychological test result of the user may include extracting a psychological test item that has been answered while the fourth biometric data inconsistent with the third biometric data is measured.

Also, a step of correcting the result of the psychological test based on a result of the analysis may include performing a calculation between a score of response of the user to the extracted psychological test item and a correction factor to adjust the response score. The psychological test may include a plurality of incompatible items or at least one item that reflects an index for estimating a response pattern of a user who makes responses distorted to conform to social norms.

Also, when the user performs a psychological test training program, the user's biometric data acquired while the program is performed (e.g., biometric data during training) may be classified based on personal information for each user or user group and stored in a database. An analysis result for such biometric data during training may be further included in the second biometric data. In other words, biometric data of a case in which the user tries to make response distortion (e.g., biometric data upon distortion) may be included in the second biometric data. Whether the user tries to make response distortion may be determined by whether there is a difference between the first biometric data and the biometric data upon distortion. For example, information regarding an increase or decrease in pulse rate, an increase or decrease in respiration rate, an increase or decrease in secretion of body fluids, an expansion or reduction of pupil, an increase or decrease in body temperature, an increase or decrease in muscle momentum, etc. may be included in the biometric data during distortion.

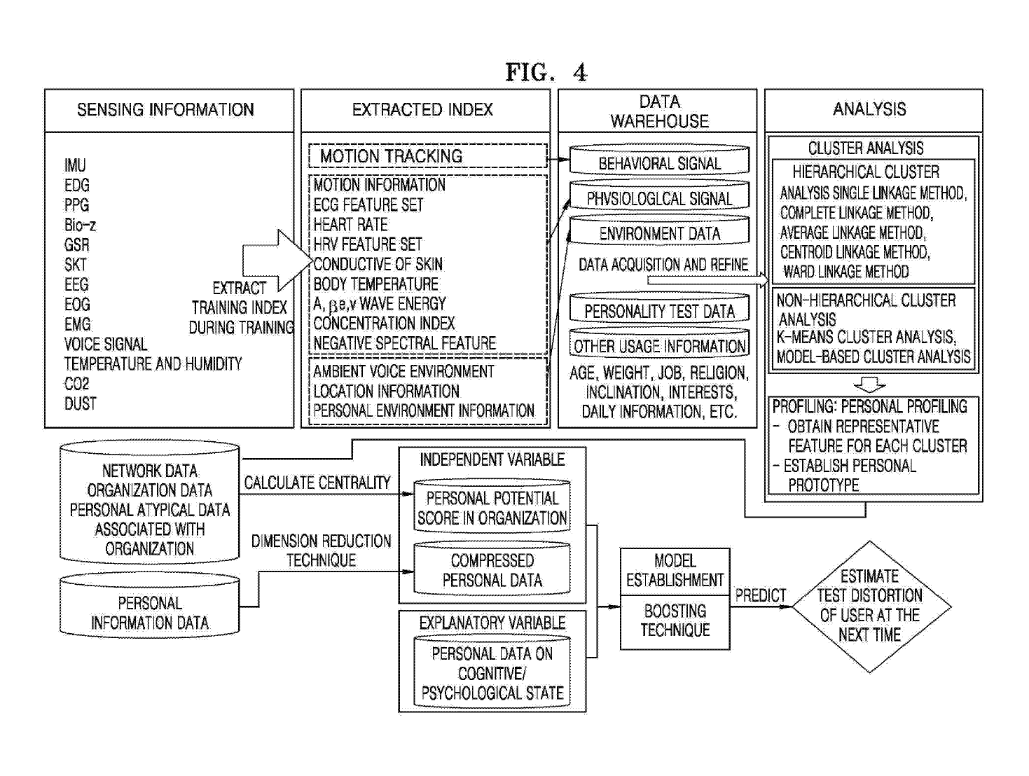

FIG. 4 is a view showing a process of estimating distortion of a psychological test using a user's biometric data according to an embodiment of the present invention.

A process of analyze features of each cluster through cluster analysis and building a personal prototype may be included in the process of estimating distortion of a psychological test using a user's biometric data according to an embodiment of the present invention.

As shown in FIG. 4, for example, a model-based cluster analysis may be performed with personal information as well as organization information and sensing information collected through a sensor. The cluster analysis may be performed as a hierarchical cluster analysis by applying an analysis technique such as a single linkage method, a complete linkage method, an average linkage method, a centroid linkage method, a Ward linkage method, etc. Alternatively, the cluster analysis may be performed as a non-hierarchical cluster analysis may be performed by applying a technique such as a K-means cluster analysis, a model-based cluster analysis, etc. According to an embodiment of the present invention, a model-based cluster analysis technique may be utilized for the cluster analysis.

Clusters may be obtained through the cluster analysis, and a representative feature for each of the clusters may be determined on the basis of information collected from individuals who belong to an identical cluster. Also, the personal prototype may be established on the basis of the representative feature determined for each cluster. The personal prototype may refer to a personal behavior trend of the user, a changing trend of biometric data in a specific environment, etc. Test distortion for the user may be estimated through the built prototype. In other words, test distortion of the user at the next time may be predicted through the built prototype, and distortion of a psychological test may be corrected by inversely applying the predicted test distortion. As an example, according to an embodiment of the present invention, a response score of the user may be reduced by reflecting the predicted test distortion in the correction factor according to an embodiment of the present invention.

Also, a psychological test distortion correction system according to an embodiment of the present invention may be provided.

The psychological test distortion correction system may include a user device 100, a psychological test device 200, and a DB server 300. The psychological test device 200 may include a psychological test distortion corrector 400.

The psychological test distortion corrector 400 may include a biometric data collection unit 410, a data analysis unit 420, and a test result correction unit 430. The biometric data collection unit 410, the data analysis unit 420, and the test result correction unit 430 may be connected to transmit and/or receive data to and/or from one another. The biometric data collection unit 410 may collect biometric data acquired or entered through the user device 100 for each user in real time. The data analysis unit 420 may perform a comparison of the biometric data and an analysis of a result of the psychological test. Also, the test result correction unit 430 may perform a calculation between a user response score for the psychological test and a correction factor.

An embodiment of the present invention may be implemented as a recording medium including computer-executable instructions such as computer-executable program module. A computer-readable medium may be any usable medium accessible by a computer and may include volatile and non-volatile media and discrete and integrated media. Also, the computer-readable medium may include both a computer storage medium and a communication medium. The computer storage medium includes the volatile and non-volatile media and the discrete and integrated media, which are implemented in any method or technique for storing information such as a computer readable instruction, data structure, program module, or other data. The communication module typically includes the computer readable instruction, data structure, program module, or other data and transmission mechanism of a modulated data signal such as a carrier and further includes any information transmission medium.

As described above, according to the one or more of the above exemplary embodiments, the method of correcting distortion of a psychological test using a user's biometric data may correct a score of a user who makes response distortion to provide a better selection that meets the objective of the psychological test.

Also, the method may train an item with a response instinctually distorted by the user through an analysis of result data of the psychological test training program, thus decreasing the response distortion of the user.

Also, the method may allow the DB server to automatically compare and read, over the Internet, biometric data obtained when the user is in a normal state, performs the psychological test training program, and the psychological test program of the user and to obtain the test result.

The above description is merely illustrative, and it will be understood by those skilled in the art that various changes in form and details may be made therein without departing from the spirit and scope of the invention as defined by the appended claims. The above embodiments are accordingly to be regarded as illustrative rather than restrictive. For example, while a single element may be distributed and then carried out, distributed elements may be carried out in a combination thereof.

Therefore, the scope of the present disclosure is defined not by the detailed description but by the appended claims, and all changes or modifications within the appended claims and their equivalents will be construed as being included in the scope of the present disclosure.

What is claimed is:

1. A method of correcting distortion of a psychological test using a user's biometric data, the method comprising:
receiving personal information for authentication from the user and performing user authentication;
acquiring first biometric data from the user at predetermined intervals while the user is in a normal state;
allowing the user to perform a psychological test training program and acquiring second biometric data from the user of the program at predetermined intervals;
comparing the first biometric data and the second biometric data to acquire third biometric data, which is reference data for distortion detection;
allowing the user to take the psychological test and acquiring fourth biometric data from the user at predetermined intervals;
comparing the third biometric data and the fourth biometric data;
analyzing a result of the psychological test of the user based on a result of the comparison; and
correcting the result of the psychological test based on a result of the analysis.

2. The method of claim 1, wherein:
the personal information includes user identification information composed of at least one of text, image, and sound;
the normal state of the user includes a state in which the user engages in daily life; and
the predetermined intervals are regular intervals of one millisecond, one second, or one minute.

3. The method of claim 2, wherein:
the personal information, the first biometric data, the second biometric data, the third biometric data, and the fourth biometric data are received or acquired through a user device;
the user device includes at least one of a data input unit and a sensor unit; and
the sensor unit includes a photoplethysmography (PPG) sensor, an electrocardiography (ECG) sensor, an electroencephalogrpahy (EEG) sensor, a galvanic skin response (GSR) sensor, a skin temperature (SKT) sensor, an accelerator, a gyro sensor, or a cortisol hormone measurement sensor.

4. The method of claim 1, wherein each of the personal information, the first biometric data, the second biometric data, the third biometric data, and the fourth biometric data includes at least one of a pulse rate, a respiration rate, secreted body fluids, a pupil size, a body temperature, and muscle momentum of the user.

5. The method of claim 1, wherein:
the psychological test training program is a program that sequentially outputs at least one of image and sound according to a psychological state of the user;
the program is temporarily or regularly performed by the user; and
the psychological state of the user is determined according to current state information entered by the user.

6. The method of claim 1, wherein:
the comparing of the third biometric data and the fourth biometric data comprises estimating a degree of consistency between the third biometric data and the fourth biometric data; and
the analyzing of a result of the psychological test of the user based on a result of the comparison comprises extracting a psychological test item that has been answered while the fourth biometric data inconsistent with the third biometric data is measured.

7. The method of claim 6, wherein:
the correcting of the result of the psychological test based on a result of the analysis comprises performing a calculation between a score of response of the user to the extracted psychological test item and a correction factor to adjust the response score; and
the psychological test includes a plurality of incompatible items or at least one item that reflects an index for estimating a response pattern of a user who makes responses distorted to conform to social norms.

8. A non-transitory computer-readable recording medium storing a program for implementing the method of claim 1.
